(19) United States
(12) Patent Application Publication
(10) Pub. No.: US 2026/0114873 A1
(43) Pub. Date: Apr. 30, 2026

(54) ENDOSCOPIC SYSTEMS AND METHODS FOR DUODENAL MUCOSAL RESHAPING AND RESURFACING

(71) Applicant: ENDOLASTIC, INC., Raleigh, NC (US)

(72) Inventors: Frederick Haller, Winston-Salem, NC (US); Paul Robertson, Raleigh, NC (US)

(21) Appl. No.: 19/366,745

(22) Filed: Oct. 23, 2025

Related U.S. Application Data
(60) Provisional application No. 63/712,093, filed on Oct. 25, 2024.

(51) Int. Cl. A61B 17/12; A61B 17/00

(52) U.S. Cl. CPC A61B 17/12013; A61B 2017/00022; A61B 2017/00238; A61B 2017/00818; A61B 2017/00893; A61B 2017/00902; A61B 2017/12018

(57) ABSTRACT

Non-resective endoscopic systems and methods for altering gastrointestinal organs to treat Type 2 Diabetes Mellitus (T2DM). The non-resective endoscopic systems and methods provide improved tissue capture to create localized pressure necrosis to target tissue areas. The non-resective endoscopic systems and methods include banding target tissue areas utilizing a ligation band. The ligation band creates a uniform distribution of pressure to improve tissue retention and pressure necrosis for eliciting a healing response.

### CROSS-REFERENCE TO RELATED APPLICATIONS

This application claims the benefit of and priority to U.S. Provisional Patent Application No. 63/712,093, filed on Oct. 25, 2024, the entire contents of which is herein incorporated by reference in its entirety.

### BACKGROUND

Type 2 Diabetes Mellitus (T2DM) is characterized by insulin resistance and inadequate insulin production, leading to hyperglycemia and related complications including cardiovascular disease, neuropathy, nephropathy, and retinopathy. T2DM can increase the negative effects of nonvascular complications including liver disease, infections, mental health disorders, cancer, and Alzheimer's disease. The duodenum is a metabolic signaling center that responds to nutrients and other substances exiting the stomach. The duodenal mucosa exhibits abnormal hypertrophy and endocrine hyperplasia when a patient is suffering from Type 2 Diabetes Mellitus. Current treatment includes lifestyle modifications, pharmacologic inventions, and bariatric surgery. Many patients struggle to achieve adequate glycemic control because of poor compliance with complex medical regimens and limited efficacy of existing therapies.

Sustaining glycemic control and mitigating diabetic complications is essential to treating T2DM. Current solutions (e.g., medications) are directed toward specific physiological targets to lower blood glucose. However, these solutions do not address the underlying pathophysiology and require continuous consumption to benefit an individual. Additionally, many individuals struggle to reach a glycemic target utilizing the current long-term therapies because of issues with adherence and access to medications. In an attempt to address the deficiency of current treatments, modifying the gastrointestinal tract has emerged as a candidate for improving the treatment of T2DM with and without medication.

Modifying the gastrointestinal tract includes altering the duodenum or removing the duodenum from intestinal nutrient flow. The duodenum is a metabolic signaling center that responds to nutrients and other substances exiting the stomach. Utilizing neural and hormonal signaling in response to nutrient absorption and sensing, the duodenum can regulate the activity of downstream organs. The neural and hormonal signaling includes appetite control, glucose production, insulin production, and gastric emptying. When a patient is suffering from T2DM, the duodenal mucosa exhibits abnormal hypertrophy and endocrine hyperplasia, which can contribute to altered nutrient sensing and hormonal signaling. Alternations of the morphology and function of the duodenal mucosa can play a critical role in the pathogenesis of T2DM. Removal of the duodenum can improve patient weight loss and glucose regulation. However, the improved glycemic control can be reverted when nutrients are reintroduced into the gastro-duodenal remnant. Therefore, there is a need for treating T2DM without removal of the duodenum.

Current DMR approaches include hydrothermal ablation and electrical ablation. These energy-based methods have inherent risks with energy application including inconsistent healing, potential for deep tissue damage, and complications such as perforation or prolonged pain. There is a need for a minimally invasive method for duodenal mucosal resurfacing and reshaping to treat Type 2 Diabetes Mellitus.

### FIELD OF THE DISCLOSURE

The present disclosure is generally directed to endoscopic systems and devices, more specifically to endoscopic systems and devices for duodenal mucosal resurfacing and reshaping via band-assisted pressure necrosis.

### BRIEF SUMMARY OF THE DISCLOSURE

Existing DMR devices and systems can cause perforation of the thin duodenum wall. Unlike existing DMR devices that use thermal or electrical energy to ablate tissue, the present disclosure utilizes controlled mechanical compression to selectively target the superficial mucosal layers while preserving deeper layers (e.g., the muscularis propria). Advantageously, the present disclosure does not involve cautery, ablation, or resection. The present disclosure utilizes controlled pressure necrosis over time, combined with simultaneous tissue regeneration. The natural tissue healing maintains the integrity of the deeper tissue layers and reduces the potential for perforation complications. Advantageously, the present disclosure results in improved glycemic control, HbA1c reduction, increased insulin sensitivity, improved incretin hormone secretion, and reduced plasma glucose levels.

The present disclosure relates to medical devices and methods for the non-surgical treatment of Type 2 Diabetes Mellitus (T2DM) through endoscopic procedures. The endoscopic procedure utilizes endoscopic barrels and a retention band for therapeutic resurfacing of the duodenal mucosa. The retention band is designed to create an optimal radial force on tissue received in the retention band to ensure proper healing while also performing safe pressure necrosis. The endoscopic procedure ensures uniform tissue compression and regeneration, thereby improving insulin sensitivity and glycemic control.

In some embodiments, an endoscopic method for duodenal mucosal resurfacing (DMR) utilizing band-assisted pressure necrosis is disclosed. The duodenal mucosal resurfacing method includes applying a plurality of ligation bands to one or more targeted areas of the duodenum of a patient to induce localized necrosis. As a result of the localized necrosis, the duodenum will undergo natural healing and tissue regeneration. The endoscopic DMR band ligation method is operable to treat duodenal varices, duodenal ulcers, neuroendocrine tumors (NETs), mucosal-based gastrointestinal stromal tumors (GISTs), angiodysplasia, and post-polypectomy bleeding.

The regenerative process after endoscopic band ligation is characterized by several stages: necrosis, inflammation, proliferation, and remodeling. The compression from ligation bands leads to localized ischemia, causing targeted tissue necrosis that triggers a controlled inflammatory response. This response facilitates the release of growth factors, such as transforming growth factor beta (TGF-β) and platelet-derived growth factor (PDGF), which recruit mesenchymal stem cells (MSCs) and fibroblasts to the site of injury. These cells play a crucial role in tissue remodeling and regeneration, supporting the restoration of a healthy mucosal layer with normal histological and functional characteristics.

Advantageously, endoscopic band ligation avoids the deep tissue damage associated with thermal ablation, reducing the risk of perforation, bleeding, or fibrosis. Endoscopic band ligation further promotes natural healing. By relying on the body's intrinsic healing mechanisms, the regeneration of functional mucosal tissue results in tissue that is less insulin-resistant. The controlled necrosis from ligation promotes a healing environment that encourages regeneration without the formation of scar tissue.

In some embodiments, an endoscope designed to secure a chamber around a targeted area of a duodenal mucosa is disclosed. The endoscope is designed to suction the mucosal tissue into an endoscopic barrel. Then, ligation bands are deployed around the captured tissue to create localized pressure zones. In some embodiments, the ligation bands are elastomeric and provided sustained, even compression over a period of time.

The controlled pressure induces localized ischemia, which leads to tissue necrosis in the targeted mucosal layers. The necrotic process initiates a highly regulated inflammatory response, promoting the release of cytokines (e.g., IL-6 and TNF-α) and growth factors (e.g., TGF-β and PDGF). These signaling molecules stimulate mesenchymal stem cells (MSCs) and fibroblasts, which are essential for tissue remodeling and regeneration. The healing cascade leads to the resurfacing of the mucosa with new, healthy tissue, thus restoring its normal functionality in glucose metabolism. Unlike thermal or electrical modalities, the endoscopic systems and methods disclosed herein minimize the risk of complications like perforation, stricture, or prolonged pain by preserving deeper tissue layers, ensuring a more predictable and safer healing process.

In some embodiments, a method for targeting duodenal mucosal surface for treating metabolic diseases without permanent implants is disclosed. In at least one aspect of the present disclosure, a band-assisted endoscopic mucosal surfacing technique involving the natural pressure-induced physical resurfacing of the superficial abnormal tissue layer, thereby promoting the regrowth of normal tissue through a stem cell mediated healing response is disclosed.

In some embodiments, a duodenal mucosal resurfacing system designed to capture tissue with a wide margin but shallow depth to ensure effective treatment while minimizing complications is disclosed. In some embodiments, the DMR system includes a 12.5-millimeter diameter endoscopic barrel for capturing a broad area of duodenal mucosa and promoting uniform resurfacing. In some embodiments, the DMR system includes a 12-millimeter diameter endoscopic barrel. The endoscopic barrel includes a 6 mm depth to reduce the risk of perforation and ensuring that deeper tissue layers, including the muscularis propria, are not involved. In one or more aspects, the endoscopic end barrel is transparent.

The present disclosure improves glycemic control by altering the duodenal mucosa and impacting glucose metabolism. The banding approach limits tissue capture depth to about 6 millimeters to reduces the risk of complication including perforations, bleeding, and post-procedural pain. The banded approach further ensures comprehensive mucosal coverage, promoting uniform tissue resurfacing and tissue regeneration. The minimally invasive nature is designed to improve recovery times and patient comfort. The banded approach is repeatable and scalable.

In some embodiments, the present disclosure includes a method for duodenal mucosal resurfacing for treating T2DM. The method includes (1) patient preparation, (2) scope insertion, (3) tissue capture, (4) band deployment, (5) band ligation, (6) repeating steps 3-5, and (7) completion and scope removal. The patient preparation step includes preparing a patient for an endoscopic procedure (e.g., conscious sedation). The scope insertion is the insertion of an upper gastrointestinal (GI) endoscope through a mouth of a patient and navigated through the pylorus, duodenal bulb arriving at the duodenum. The tissue capture step includes endoscopically placing an endoscopic barrel at treatment sites along a duodenal wall and suction is applied to the tissue to capture the tissue. In some embodiments, the present disclosure includes determining a target area of the duodenum based on (1) anatomical landmarks, (2) treatment length, (3) tissue layers, and (4) patient safety. For example, and without limitation, the DMR method includes beginning distally of the ampulla of Vater to avoid complication related to bile and pancreatic fluid flow. The treatment area extends toward the ligament of Treitz. For further example, in some embodiments, about 4 centimeters of the duodenum is resurface. In some embodiments, up to about 9.5 centimeters of the duodenum is resurfaced. In some embodiments, only the duodenal mucosa is captured.

In some embodiments, the DMR method includes injecting saline to create a protective barrier near the duodenum. The protective barrier separates the mucosal and submucosal layers from deeper muscle layers, provides a protective fluid cushion, reduces perforation and thermal injury risk, and facilitates safer tissue removal and treatment. In some embodiments, glycerol mixtures and other viscous solutions are used to create the protective barrier.

In some embodiments, the band deployment includes ligation bands for capturing and securing the duodenal mucosa with a 12.5 mm diameter barrel. The band ligation includes ligated the captured mucosa using ligation bands to apply controlled pressure to promote tissue resurfacing and regeneration. The steps are repeated until the entire desired duodenal surface area is treated. The devices are carefully removed and a patient is monitored for immediate post-procedural complications.

In some embodiments, the ligation band allows tissue to extrude through gaps in the side walls of the band. This creates a mechanical lock as the tissue expands into the space, thereby preventing the ligation band from slipping. The pressure exerted by the ligation band is from both an external surface and internal surface on the tissue that passes through the gaps of the ligation band. As a result, the pressure force is distributed across the tissue and reduces the risk of tissue damage or necrosis that typically occurs because of concentrated pressure points. By allowing the tissue to fill the gaps, the ligation band is adaptable for numerous environments and procedures. The reduced tissue slippage, improved tissue retention, and natural pressure distribution of the ligation band reduces tissue trauma, thereby improving recovery outcomes and lowering the risk of complications (e.g., inflammation or infection).

In some embodiments, a duodenal mucosal resurfacing endoscopic system is disclosed. The duodenal mucosal resurfacing system is designed to capture and retain large tissue volumes (e.g., at least 625 mm³) via non-resection banding. The duodenal mucosal resurfacing endoscopic system applies pressure necrosis while capturing a targeted area of the duodenum.

In some embodiments, the duodenal mucosal resurfacing endoscopic system includes an endoscopic barrel for T2DM. The T2DM endoscopic barrel includes a diameter of about 12.5 mm, a depth of about 6 mm, and a volume of about 625 mm³. In some embodiments, the duodenal mucosal resurfacing endoscopic barrel includes a diameter of about 15 mm, a depth of about 6 mm, and a volume of about 1050 mm³.

In some embodiments, the duodenal mucosal resurfacing endoscopic device includes a medical device with anti-slippage properties that is designed to apply uniform pressure to a target area of tissue. The medical device is operable for duodenal mucosal resurfacing and includes an endoscopic barrel and a ligation band. The endoscopic barrel includes a volume that is sufficient to capture multiple layers of tissue. The ligation band is operable for band-assisted non-resective modification of gastrointestinal organs. The duodenal mucosal resurfacing endoscopic device increases the tissue capacity within the endoscopic barrel, thereby enhancing the resurfacing effects without surgical resection.

In some embodiments, a method for treating a patient by resurfacing a body lumen or passageway without tissue removal is disclosed. The method includes banding and capturing the submucosal and mucosal layers to create a fibrotic effect to alter the duodenum. Advantageously, the method does not include resection or removal of tissue and eliminates the risk of perforation of the muscle wall.

In some embodiments, the method includes banding an amount of tissue from a body lumen wall and/or banding tissue at a treatment site to evoke an injurious stimulus to create a tissue healing response. In some embodiments, the banded tissue is limited to tissue that occurs on a luminal side of a passageway wall. For example, and without limitation, the lumen walls of body passageways include the duodenum. These lumen walls include a mucosal layer and an underlying submucosal layer. The method includes banding the mucosal layer and/or the submucosal layer without causing damage to the underlying muscle layer. In some embodiments, the underlying muscle layer is also banded.

In some embodiments, the present disclosure includes a method for treating a patient by resurfacing a body lumen or passageway. The method includes selective tissue banding, which, upon healing, results in resurfacing. The methods include treatment of a duodenum to improve glycemic control. In some embodiments, the method includes identifying a reduction of a body passageway that will reduce flow through the corresponding body passageway to improve a patient's condition. The method further includes banding tissue from an inner portion of a wall defining the identified body passageway to elicit the healing response.

In some embodiments, a method of ligating tissue using a ligation band is provided. The method of ligating tissue includes pulling tissue into an inner lumen of a barrel. The ligation band is moved along the barrel until the ligation band is positioned around targeted tissue. The ligation band grips the enclosed tissue and provides anti-slipping functionality.

In some embodiments, a ligation band is used in combination with an endoscopic band gastroplasty (EBG) barrel. The EBG barrel includes a diameter of about 15 millimeters (mm), a captured tissue depth of about 6 mm, a surface area of tissue of about 177 mm², and a captured tissue volume of about 1,050 mm³. In some embodiments, the barrel includes a 12.5 mm diameter and a tissue volume of 625 mm³.

In some embodiments, an endoscopic system for duodenal mucosal surface resurfacing is disclosed. The endoscopic system includes an endoscope including at least one end barrel and at least one elastomeric ligation band. The end barrel includes a clear, plastic material and a diameter of at least 9.2 millimeters and a depth of at least 6 millimeters. The endoscope captures tissue corresponding to a patient. The tissue includes a mucosal layer and a submucosal layer. The at least one elastomeric ligation band is operable to band the mucosal layer and the submucosal layer. The banding of the mucosal layer and the submucosal layer generates a controlled pressure across the banded tissue, thereby inducing ischemia followed by pressure necrosis and subsequent fibrosis. As a result, natural healing response is generated without the need for resection, ablation, cryotherapy, argon plasma coagulation (APC), radiofrequency ablation (RFA), suturing, stapling, clipping, or other invasive modalities.

In some embodiments, a method for treating gastrointestinal tissue is disclosed. The method for treating gastrointestinal tissue includes (1) identifying a body passageway for narrowing for resurfacing to perform duodenal mucosal resurfacing (DMR) to treat Type 2 Diabetes Mellitus or perforation closure, (2) capturing, via an endoscopic device, at least one tissue area corresponding to the identified body passageway or tissue defect, (3) deploying at least one elastomeric ligation band to the at least one tissue area, and (4) banding via the at least one elastomeric ligation band, the at least one tissue area, wherein the at least one tissue area undergoes pressure necrosis to elicit a natural healing response without resection or ablation.

In some embodiments, a method for resurfacing gastrointestinal tissue to perform duodenal mucosal resurfacing is disclosed. The method for performing duodenal mucosal resurfacing includes removing an insulin-resistant mucosal layer and promoting regeneration without narrowing the body passageway. Typical ablation methods include mucosal layer and risk damage to deeper layers of tissue. The implementation of banding as taught herein provides controlled, localized, tissue regeneration and lowers the risk of deep tissue damage.

In some embodiments, a method for treating perforation closure is disclosed. The method for treating perforation closure includes banding the mucosal layer and the submucosal layer to close and seal perforation and regenerate healthy tissue. In some embodiments, an endoscopic device captures at least one tissue area via suction. In some embodiments, the natural healing response improves a patient's condition.

In some embodiments, an endoscopic banding system for duodenal mucosal resurfacing (DMR) to treat Type 2 Diabetes Mellitus (T2DM) utilizing elastic band ligation to treat targeted tissue areas effectively is disclosed. Advantageously, the endoscopic banding system does not use resection. The endoscopic banding system includes a plurality of ligation bands. The targeted tissue is drawn into the endoscopic barrel by suction created by a suction channel in the endoscope. The suctioned tissue is banded and, as a result, blood supply and nutrients to the banded tissue is restricted, which causes ischemia. Prolonged ischemia can lead to necrosis. Necrosis typically starts within twenty-four (24) hours of restricted blood flow to the banded tissue. Necrosis is characterized by the irreversible damage and death of the cells in the tissue. The necrotic tissue sloughs off and fibroblasts and stem cells infiltrate the banded tissue to promote fibrosis and connective tissue formation. Next, re-epithelization (cell remodeling) occurs and leads to healthy tissue regeneration moderated by stem cell activity. Full re-epithelialization and tissue regeneration can occur after about 21 days.

In some embodiments, duodenal mucosal resurfacing for treating Type 2 Diabetes Mellitus is performed by resurfacing the mucosal lining of the duodenum to improve insulin sensitivity and glucose metabolism.

Advantageously, the systems and methods disclosed herein are an improvement over current techniques because (1) the exclusion of muscle fibers, (2) reduced risk, (3) enhanced healing, and (4) enhanced stability and durability. The lack of resection reduces the risk of bleeding and perforation associated with cutting muscle fibers. A natural healing process occurs, lowering the perforation risk and improving safety.

In some embodiments, the present disclosure includes a method of duodenal mucosal resurfacing for treating metabolic diseases without permanent implants. The band-assisted endoscopic mucosal ligation includes pressure-induced physical resurfacing of the superficial abnormal tissue layer, promoting the regrowth of normal tissue through a stem cell-mediated healing response. An area of the duodenal mucosa is captured via an endoscopic barrel (e.g., 12 mm, 12.5 mm diameter barrel), promoting uniform resurfacing, while the minimal depth (e.g., 6 mm) reduces the risk of perforation and ensures that deeper tissue layers (e.g., muscularis propria) are not involved. Advantageously, the disclosed endoscopic systems and methods reduce the risk of complications such as perforation, bleeding, and post-procedural pain because of the limited tissue captured depth. The present disclosure promotes uniform tissue resurfacing and tissue regeneration. The minimally invasive nature of the duodenal mucosal resurfacing leads to shorter recovery times and improved patient comfort.

In some embodiments, a method of duodenal mucosal resurfacing is disclosed. The method of duodenal mucosal resurfacing includes preparing a patient for an endoscopic procedure. An upper GI endoscope is inserted through the patient's mouth and navigated through the pylorus, duodenal bulb, arriving at the duodenum. The endoscope includes a barrel that is placed at treatment sites along the duodenal wall and suction is applied to the treatment site tissue to capture the tissue into the barrel. At least one ligation band is deployed to capture and secure the duodenal mucosa within the endoscopic barrel. After banding, the captured tissue undergoes pressure to promote tissue resurfacing and regeneration. In at least one aspect, the at least one ligation band sloughs off of the banded tissue between about one to three weeks.

### BRIEF DESCRIPTION OF THE DRAWINGS

The embodiments illustrated, described, and discussed herein are illustrative of the present disclosure. As these embodiments of the present disclosure are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present disclosure, and through which these teachings have advanced the art, are considered to be within the spirit and scope of the present disclosure. Hence, these descriptions and drawings should not be considered in a limiting sense, as it is understood that the present disclosure is in no way limited to only the embodiments illustrated.

FIG. 1 illustrates an anatomical view of a portion of a gastrointestinal tract.

FIG. 2 illustrates a ligation band according to one embodiment of the present disclosure.

FIG. 3A illustrates a detailed view of a ligation band according to one embodiment of the present disclosure.

FIG. 3B illustrates a detailed view of a ligation band according to one embodiment of the present disclosure.

FIG. 3C illustrates a detailed view of a ligation band according to one embodiment of the present disclosure.

FIG. 3D illustrates a detailed view of a ligation band according to one embodiment of the present disclosure.

FIG. 4 illustrates banding of a pseudopolyp according to one embodiment of the present disclosure.

FIG. 5 illustrates a DMR system according to one embodiment of the present disclosure.

FIG. 6 illustrates banding of tissue utilizing a DMR system according to at least one embodiment of the present disclosure.

FIG. 7 illustrates an interior view of a portion of a duodenum.

FIG. 8 illustrates banding of duodenal tissue using a DMR system according to at least one aspect of the present disclosure.

FIG. 9 illustrates banding of duodenal tissue using a DMR system according to at least one aspect of the present disclosure.

### DETAILED DESCRIPTION

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a,” “an,” and “the” refer to “one or more” when used in the subject specification, including the claims. Thus, for example, reference to “a device” can include a plurality of such devices, and so forth.

Unless otherwise indicated, all numbers expressing quantities of components, conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

Non-resective endoscopic systems and methods for altering gastrointestinal organs to treat Type 2 Diabetes Mellitus (T2DM) are disclosed herein. The non-resective endoscopic systems and methods provide improved tissue capture to create localized pressure necrosis to target tissue areas. The non-resective endoscopic systems and methods include banding target tissue areas utilizing a ligation band. The ligation band creates a uniform distribution of pressure to improve tissue retention and pressure necrosis for eliciting a healing response.

In some embodiments, a duodenum resurfacing endoscopic system including an endoscopic ligator barrel and at least one ligation band is disclosed. The endoscopic ligator barrel is operable to receive the at least one ligation band. In some embodiments, a pull line is operable to deploy the one or more ligation bands. The gastrointestinal resurfacing endoscopic system further includes an endoscope.

In some embodiments, the gastrointestinal resurfacing endoscopic system is operable for narrowing a body passageway. The narrowing of the body passageway includes (1) identifying a body passageway corresponding to a patient's medical condition, (2) determining that narrowing of a luminal diameter of the corresponding body passageway will decrease flow through the corresponding passageway, (3) determining that the decreased flow will provide relief and/or improve a patient's medical condition, (4) determining an amount of reduction to provide effective treatment, and (5) using an endoscopic device to band a portion of the luminal surface of the corresponding body passage to elicit a healing response. The method for narrowing a body passageway does not resect or remove tissue, thereby reducing risk associated with cutting and removing tissue.

In some embodiments, the duodenum resurfacing endoscopic system is operable to band patient tissue using an endoscope and a ligation band to modify a portion of a duodenum of a patient to improve glycemic control. In some embodiments, the duodenum resurfacing endoscopic system includes conducting an endoscopic procedure that includes banding a patient's duodenum to improve glycemic control.

In some embodiments, a method for narrowing a body passageway is disclosed. The method includes (a) determining a first treatment area along a body passageway, (b) isolating a first portion of the mucosa and submucosa using an endoscopic device, (c) banding the first portion of the mucosa and submucosa layer, (d) moving circumferentially of the body passageway adjacent to the first banded portion, to a second portion, and (e) banding the second portion of the mucosal and submucosal layers.

In some embodiments, the method for narrowing a body passageway bands luminal tissue to create a pressure area that promotes healing of the luminal tissue. The narrowing of the body passageway includes decreasing a diameter of the body passageway, creating folds or irregular surfaces, and/or altering the contour of a body lumen wall.

In some embodiments, banding an amount of tissue in at least one body lumen wall to resurface a target body lumen is disclosed. For example, and without limitation, in some embodiments, at least two target tissue areas are banded. In some embodiments, the target tissue areas are separated by undisturbed tissue. In some embodiments, the target tissue areas overlap. The banding of the tissue areas results in a decrease in the diameter of a body lumen passageway. In some embodiments, the body lumen passageway diameter is reduced at least ten percent.

For example, and without limitation, in some embodiments, the present disclosure includes a procedure for banding tissue in a first tissue area and a second tissue area. The amount of banded tissue can be the same. In some embodiments, the amount of banded tissue varies for each tissue area. For further example, and without limitation, in some embodiments, a first banded tissue area is between about 1% and about 50% greater than a second banded tissue area. In some embodiments, determining a target area of the duodenum based on (1) anatomical landmarks, (2) treatment length, (3) tissue layers, and (4) patient safety is disclosed. For example, and without limitation, the DMR method includes beginning distally of the ampulla of Vater to avoid complications related to bile and pancreatic fluid flow. The treatment area extends toward the ligament of Treitz. For further example, in some embodiments, about 4 centimeters of the duodenum is resurfaced. In some embodiments, up to about 9.5 centimeters of the duodenum is resurfaced. In some embodiments, only the duodenal mucosa is captured.

The ligation band can be used in combination with an endoscope to capture and band tissue. In some embodiments, suction is provided and draws the mucosal layer and the submucosal layer to the ligation band. The ligation band is deployed and bands the captured tissue. In some embodiments, a plurality of ligation bands is used. The ligation band is capable of capturing and retaining a mucosal layer and/or a submucosal layer of a duodenum for pressure necrosis for the purpose of promoting healthy tissue generation.

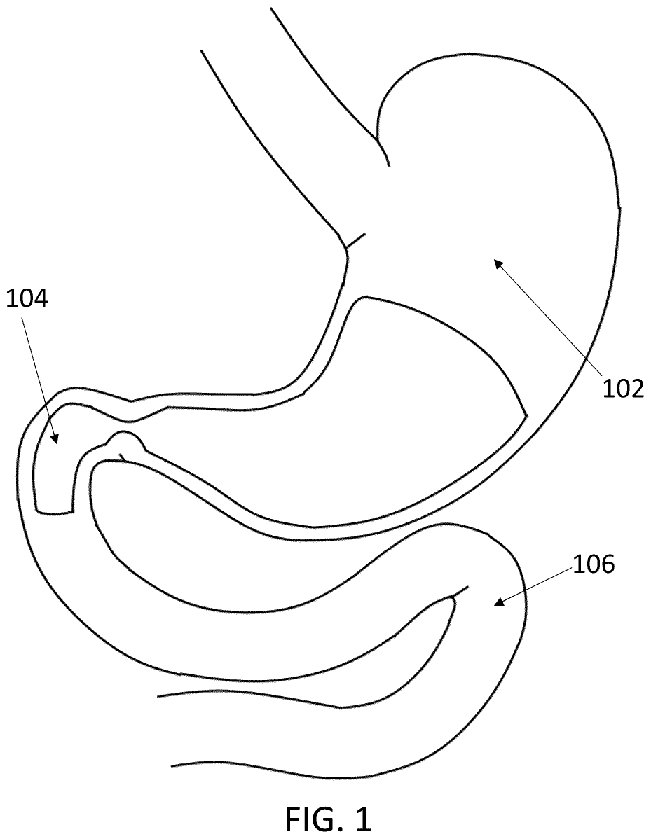

FIG. 1 illustrates a stomach 102, a duodenum 104, and a small intestine 106 of a gastrointestinal tract. For example, and without limitation, in some embodiments, the present disclosure includes a procedure and system to resurface and narrow a portion of the duodenum. Advantageously, the resurfacing and narrowing of the body passageway in the duodenum can improve glycemic control.

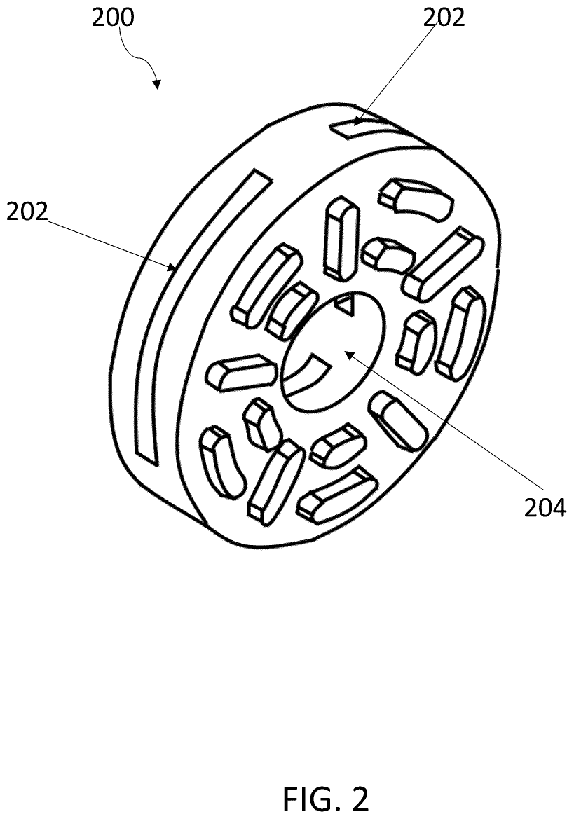

In some embodiments, as shown in FIG. 2, a ligation band 200 is disclosed. The ligation band 200 includes a plurality of channels 202 and at least one hole 204. The plurality of channels 202 includes at least three channels. In some embodiments, the plurality of channels includes a tapered shape, a curved shape, a rectangular shape, and other polygonal shapes or surface modifications increasing friction. The plurality of channels 202 is connected to the at least one hole 204. For example, and without limitation, each channel of the plurality of channels is connected to the at least one hole at about halfway between the top and bottom of the at least one hole. Each channel of the plurality of channels is designed to receive tissue that passes through the at least one hole 204. For example, and without limitation, the plurality of channels is operable to mechanically lock tissue received from the at least one hole. For further example, tissue can squeeze through the plurality of channels and press against all walls of a channel. The at least one hole 204 runs (e.g., from top to bottom) through a central portion of the ligation band 200. Advantageously, when performing ligation, the at least one hole 204 passes compressed tissue through the ligation band 200. The ligation band 200 is operable to generate a uniform force of pressure to the captured tissue to improve pressure necrosis and a corresponding healing response.

In some embodiments, the plurality of channels includes at least one gripping feature. The at least one gripping feature is designed to frictionally hold tissue. The at least one gripping feature can be a cylindrical shape, a rectangular shape, a conical shape, and other similar polygonal shapes. In some embodiments, the ligation band includes a rough surface for frictionally holding tissue. In some other embodiments, the ligation band includes a rough surface and discreet gripping features.

In some embodiments, each channel is equally spaced apart. Alternatively, the plurality of channels is not equally spaced part. In some embodiments, the ligation band further includes a plurality of side channels. The plurality of side channels is connected to a central hole of the ligation band and enables the tissue to extend to the side of the ligation band. For further example, and without limitation, each side channel of the plurality of side channels is vertically adjacent to each other and connected to the same side of the hole. In some embodiments, vertically adjacent side channels are relative to the height of the ligation band.

In some embodiments, the ligation band is a unitary piece. Alternatively, the ligation band is at least two separate pieces that are removably attached to each other. For example, and without limitation, a bottom component of the ligation band includes a plurality of holes for receiving an attachment component (e.g., pin) of a top component of the ligation band. Alternatively, or additionally, a top component and a bottom component of a ligation band are adhesively attachable. In some embodiments, the plurality of protrusions is positioned on both a top and bottom of the ligation band.

In some embodiments, a ligation band designed for minimizing slippage and retaining tissue for pressure necrosis is disclosed. The ligation band captures tissue and retains the tissue in place for a duration of time. For example, and without limitation, the plurality of channels is operable to mechanically lock tissue received from the at least one hole. For further example, tissue can squeeze through the plurality of channels and presses against all walls of a channel. The depth and width of tissue above the band and inside tissue receiving channels increases the potential for a more effective mechanical lock. The ligation band is designed to generate an adequate compression force to assist with the capture and retention of tissue. The ligation band includes surface elements to assist with anti-slippage. In some embodiments, the ligation band further includes a plurality of gripping features designed to improve the capture and retention of tissue by the ligation band.

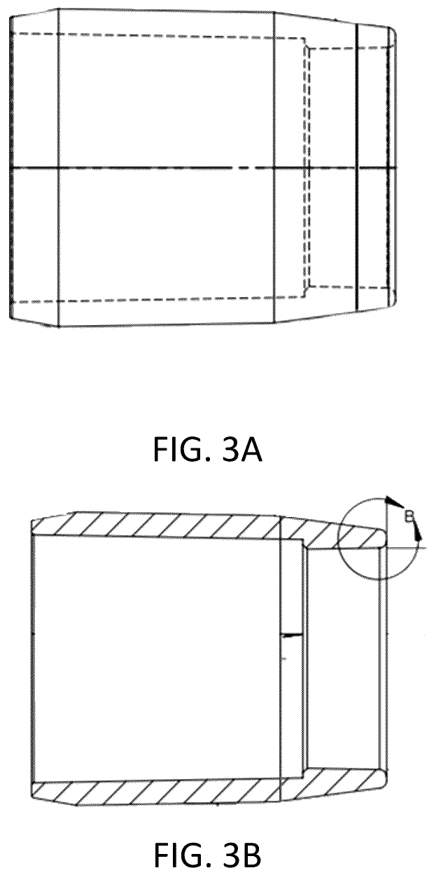

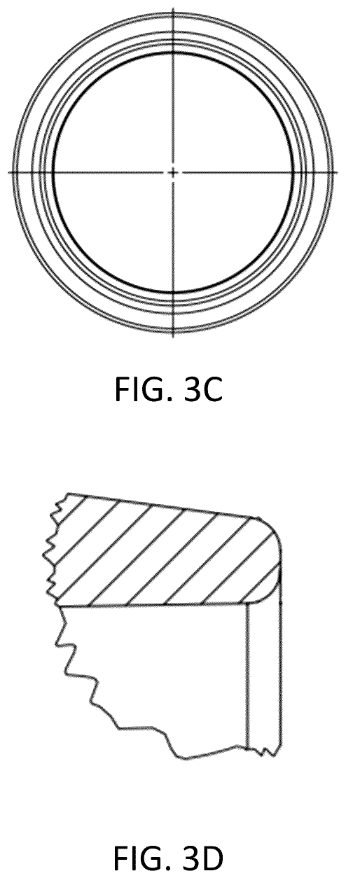

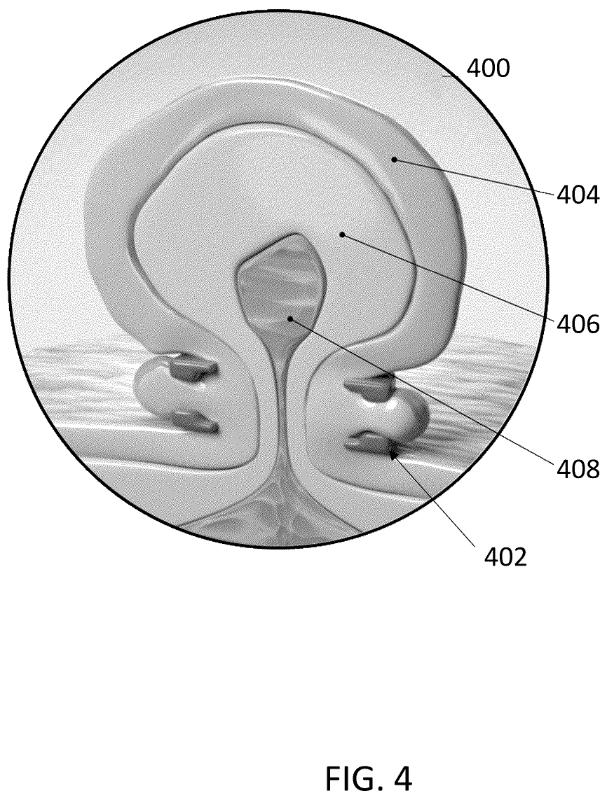

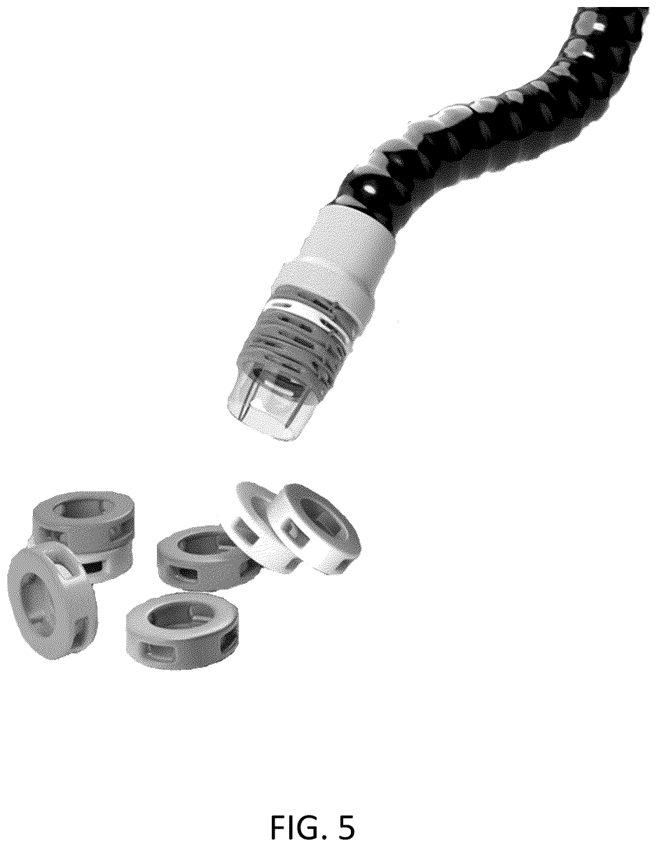

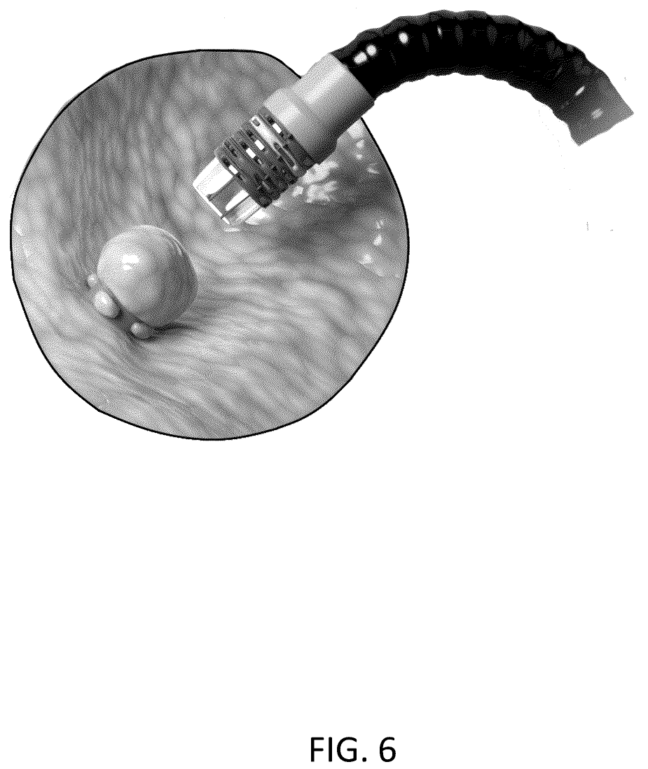

FIGS. 3A-3D illustrate detailed views of a ligation band according to at least one aspect of the present disclosure. In some embodiments, as shown in FIG. 4, a pseudopolyp 400 is banded by a ligation band 402. The pseudopolyp may include a mucosal layer 404, a submucosal layer 406, and a muscularis propria layer 408. As shown in FIGS. 5 and 6, one or more ligation bands can be used in combination with an endoscope to capture and band tissue. In some embodiments, suction is provided and draws the mucosal layer of a duodenum. In some embodiments, the mucosal layer and the submucosa layer are suctioned. The ligation band is deployed and bands the captured tissue. Notably, the muscularis propria is not banded to avoid damaging the muscle layer. Advantageously, the banded tissue undergoes controlled pressure necrosis over time, resulting in natural tissue regeneration. The natural tissue healing maintains the integrity of the deeper tissue layers and reduces the potential for perforation complications.

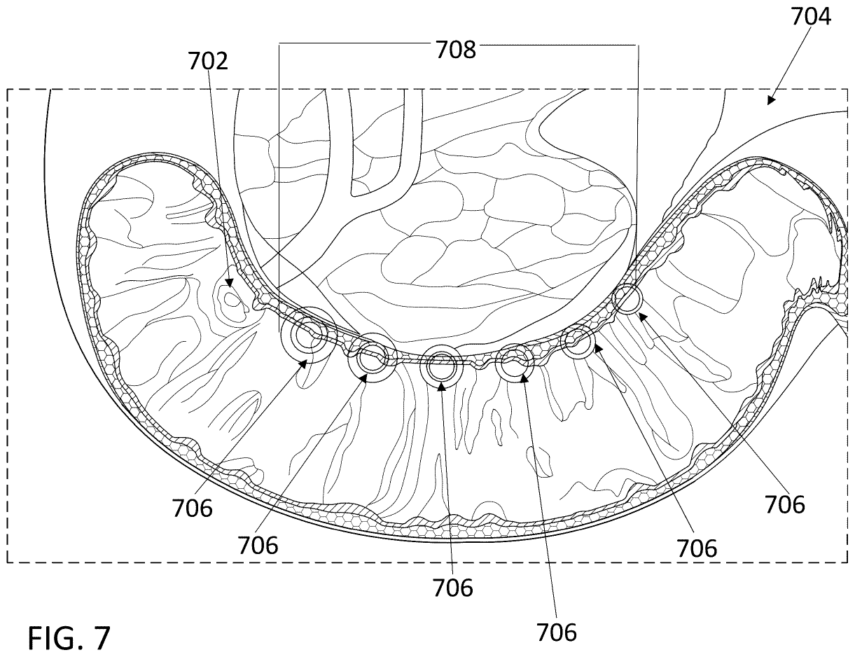

FIG. 7 illustrates an interior perspective of a duodenum. In at least one aspect of the present disclosure, a DMR system and method for resurfacing the duodenum is disclosed. Beginning distally of the ampulla of Vater 702 and extending to the ligament of Treitz 704, one or more target areas 706 of duodenal tissue is captured with an endoscope and one or more ligation bands. The one or more target areas 706 can be any section of duodenal tissue. For example, and without limitation, the one or more target areas may be positioned on the mucosal submucosa, muscularis, or serosa layers. For further example, in some embodiments, a resurface treatment length 708 is about 4 centimeters of the duodenum. In some embodiments, a resurface treatment length 708 is up to about 10 centimeters of the duodenum. In some embodiments, only the duodenal mucosa is captured.

Once the tissue is captured, one or more ligation bands is deployed to create a localized pressure zone. As a result, blood supply and nutrients to the banded tissue is restricted, which causes ischemia. Prolonged ischemia can lead to necrosis. Advantageously, the banded tissue undergoes controlled pressure necrosis over time, resulting in natural tissue regeneration. The natural tissue healing maintains the integrity of the deeper tissue layers and reduces the potential for perforation complications.

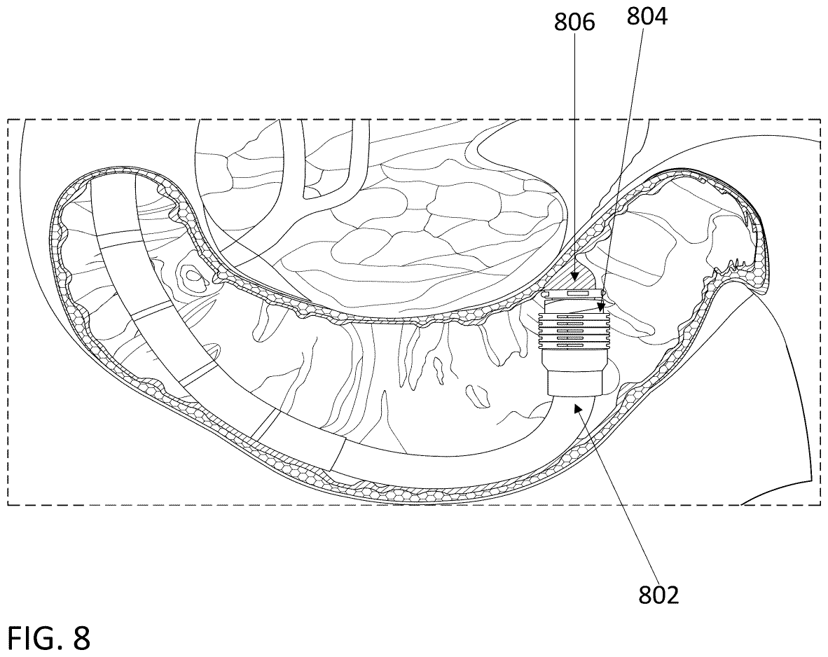

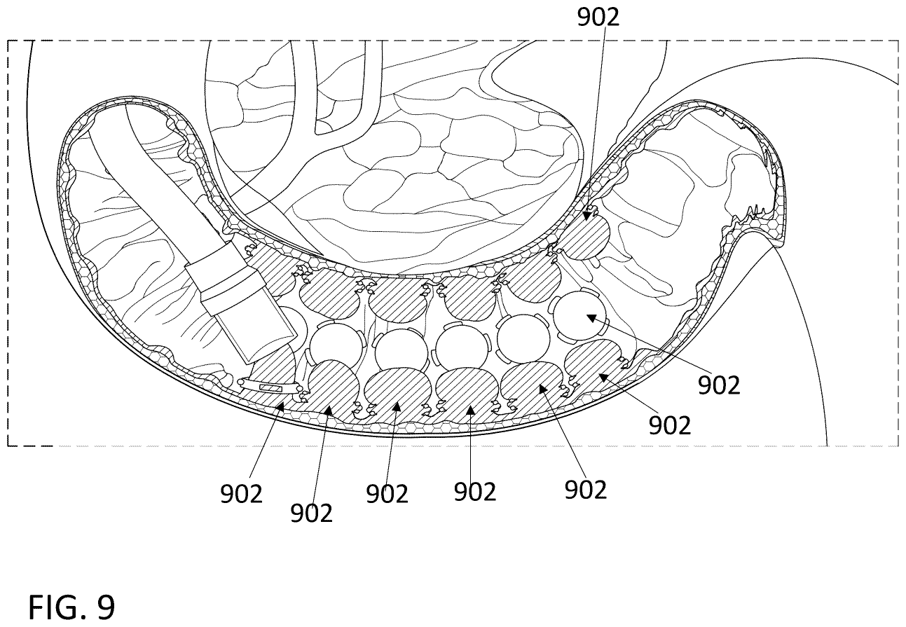

As shown in FIG. 8, a DMR system including an endoscope 802 with a plurality of ligation bands 804 is disclosed. The endoscope 802 suctions target duodenal tissue into an endoscopic barrel. The target duodenal tissue may be positioned between the Ampulla of Vater and the Ligament of Treitz. The plurality of ligation bands 804 is deployed around the captured tissue to create one or more localized pressure zones 806. A pseudopolyp may be formed. The pseudopolyp may include at least one of a mucosal layer, a submucosal layer, and a muscularis propria layer. In at least one aspect, the muscularis propria layer is not captured to avoid damaging the muscle layer. FIG. 9 illustrates the capture and banding of a plurality of target tissue areas 902. Advantageously, the banded tissue undergoes controlled pressure necrosis over time, resulting in natural tissue regeneration. The natural tissue healing maintains the integrity of the deeper tissue layers and reduces the potential for perforation complications.

The corresponding structures, materials, acts, and equivalents of all means or step plus function elements in the claims below are intended to include any structure, material, or act for performing the function in combination with other claimed elements as specifically claimed.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

What is claimed is:

1. An endoscopic system for duodenal mucosal resurfacing, the endoscopic system comprising:
an endoscope including at least one end barrel; and
at least one ligation band;
wherein the endoscope is operable to capture tissue corresponding to a patient,
wherein the captured tissue includes a mucosal layer; and
wherein the at least one ligation band is operable to band the mucosal layer; and
wherein, once the mucosal layer is banded, the at least one ligation band generates pressure across the banded tissue, wherein the banded tissue undergoes pressure necrosis to elicit a healing response.

2. The endoscopic system of claim 1, wherein the at least one ligation band further bands a submucosal layer.

3. The endoscopic system of claim 1, wherein the captured tissue includes tissue corresponding to a duodenum of the patient.

4. The endoscopic system of claim 3, further comprising a submucosal injection mechanism, wherein the submucosal injection mechanism injects a protective barrier around the duodenum.

5. The endoscopic system of claim 1, wherein the healing response includes tissue regeneration, wherein the healing response treats Type 2 Diabetes Mellitus.

6. The endoscopic system of claim 1, wherein the at least one end barrel includes a diameter of at least 8 millimeters and a depth of at least 5 millimeters.

7. A method for treating Type 2 Diabetes Mellitus comprising:
identifying at least one target area in a duodenum of a patient;
capturing, via an endoscopic device, the at least one target area;
deploying at least one elastomeric ligation band to a portion of the at least one target area; and
banding, via the at least one elastomeric ligation band, the portion of the at least one target area, wherein the at least one target area undergoes pressure necrosis to elicit a healing response, wherein the healing response includes tissue regeneration of the duodenal tissue to improve insulin sensitivity.

8. The method of treating Type 2 Diabetes Mellitus of claim 7, wherein the at least one target area includes a surface area of at least five centimeters of duodenal tissue.

9. The method of treating Type 2 Diabetes Mellitus of claim 7, wherein the at least one elastomeric ligation band includes a plurality of elastomeric ligation bands, wherein the plurality of elastomeric ligation bands is deployed along a length of the at least one target area.

10. The method of treating Type 2 Diabetes Mellitus of claim 7, wherein the at least one elastomeric ligation band sloughs off of the banded tissue between about one to three weeks.

11. A method of modifying duodenal mucosal tissue to treat Type 2 Diabetes Mellitus comprising:
inserting an endoscopic device into a duodenum corresponding to a patient;
identifying at least one target area for tissue modification;
capturing, via the endoscopic device, at least a portion of the at least one target area of the duodenum;
deploying at least one elastomeric ligation band to the portion of the at least one target area; and
banding, via the at least one elastomeric ligation band, the portion of the at least one target area; wherein the at least one target area undergoes pressure necrosis to elicit a natural healing response, wherein, as a result of the banding, tissue sloughs off the at least one target area, wherein the healing response includes tissue regeneration to improve insulin sensitivity and glucose metabolism.

12. The method of claim 11, further comprising monitoring glycemic control.

13. The method of claim 11, wherein the regenerated duodenal tissue includes improved incretin hormone secretion.

14. The method of claim 11, wherein the controlled pressure necrosis does not affect a muscularis propria layer of the duodenum.

15. The method of claim 11, wherein the endoscopic device includes at least one transparent end barrel.
